(19) United States
(12) Patent Application Publication
(10) Pub. No.: US 2025/0292410 A1
(43) Pub. Date: Sep. 18, 2025

(54) METHOD AND APPARATUS FOR CREATING A CARDIAC CONTOUR PREDICTION MODEL, AND METHOD AND SYSTEM FOR DETERMINING CARDIAC HYPERTROPHY IN ANIMALS USING THE SAME

(71) Applicant: SEOUL NATIONAL UNIVERSITY R&DB FOUNDATION, Seoul (KR)

(72) Inventors: Joohon SUNG, Yongin-si (KR); Yeojin JEONG, Seoul (KR)

(21) Appl. No.: 19/220,931

(22) Filed: May 28, 2025

Related U.S. Application Data
(63) Continuation of application No. PCT/KR2023/019680, filed on Dec. 1, 2023.

(30) Foreign Application Priority Data
Dec. 2, 2022 (KR) 10-2022-0166576

(51) Int. Cl. G06T 7/12; G06T 7/00; G06V 10/774; G06V 10/776

(52) U.S. Cl. CPC G06T 7/12; G06T 7/0012; G06V 10/774; G06V 10/776; G06T 2207/10116; G06T 2207/20021; G06T 2207/20072; G06T 2207/20081; G06T 2207/30012; G06T 2207/30048

(57) ABSTRACT

According to one embodiment, a method of generating a cardiac contour prediction model is provided. The method is performed by a processor and comprises: a step of preprocessing chest radiographic images of animals and labeled images including a cardiac contour image and a vertebral body contour image of a specific thoracic vertebral region, thereby generating a training dataset; and a step of generating an artificial intelligence model trained to output a cardiac contour image and a vertebral body contour image of the specific thoracic vertebral region of a subject animal from a chest radiographic image of the subject animal, based on the training dataset.

### CROSS-REFERENCE TO RELATED APPLICATION

This application is a continuation application of PCT International Application No. PCT/KR2023/019680 filed on Dec. 1, 2023, which claims priority to Korean Patent Application No. 10-2022-0166576, filed on Dec. 2, 2022, in the Korean Intellectual Property Office, the disclosure of which is incorporated by reference herein in its entirety.

### BACKGROUND

### 1. Field

The present invention relates to a method and apparatus for creating a cardiac contour prediction model, and a method and system for determining cardiac hypertrophy in animals using the same, and more particularly, to a method and apparatus for generating an artificial intelligence model trained to output a cardiac contour image and a vertebral body contour image of a specific thoracic vertebral region of a subject animal from a chest radiographic image of the subject animal, based on a training dataset, and to a method and system for determining cardiac hypertrophy in animals using the same.

### 2. Description of the Related Art

As lifestyles have changed due to declining birthrates, aging populations, and the increase in single-person households, the number of people raising companion animals such as dogs and cats has been increasing. In order to maintain the health of their companion animals, pet owners regularly visit animal hospitals to prevent diseases such as cardiovascular conditions. Among cardiovascular diseases in dogs, myxomatous mitral valve disease (MMVD) is the most common disease worldwide. MMVD is a condition in which the mitral valve of the heart fails to close properly, and due to mitral degeneration, blood regurgitation occurs, leading to gradual enlargement of the left atrium and left ventricle. MMVD is classified into stages A through D according to the progression of the disease. Stage A indicates no clinical symptoms but a predisposition to risk factors. Stage B is subdivided into stage B1, where there is blood regurgitation due to mitral degeneration but no cardiac enlargement, and stage B2, where blood regurgitation exists and cardiac enlargement is present. Stage C is characterized by clinical symptoms in the presence of blood regurgitation and cardiac enlargement. Stage D refers to a state in which symptoms repeatedly recur despite medical treatment in stage C. Since medical treatment is recommended from stage B2, it is extremely important to detect the progression of the disease from stage B1 to B2 and begin medication at an appropriate time to improve survival rates. Therefore, in MMVD, the necessity of medical intervention is determined based on the presence of cardiac hypertrophy.

Conventional methods for diagnosing cardiac hypertrophy in animals with MMVD include a method of measuring cardiac hypertrophy using echocardiography in companion animals such as dogs and cats, and a method of measuring cardiac hypertrophy using vertebral heart scale (VHS) based on chest radiographic images. The method using echocardiography determines the presence of cardiac hypertrophy based on the ratio of the diameters of the left atrium to the aorta measured in the echocardiographic image, or based on a value obtained by correcting the internal diameter of the dilated left ventricle according to the body weight of the animal.

The VHS method is based on research findings that the vertebral body length of the thoracic spine in dogs and cats has a linear relationship with heart size regardless of breed or body type. The VHS method determines cardiac hypertrophy by adding the long-axis and short-axis lengths of the heart from a chest radiographic image of an animal such as a dog or cat, and converting this sum into the number of thoracic vertebral bodies starting from the fourth thoracic vertebra. The long axis of the heart is the distance from the tracheal bifurcation to the cardiac apex. The short axis of the heart is perpendicular to the long axis and is the length of the line starting from the dorsal border of the caudal vena cava.

Although the echocardiographic method is the most accurate for determining cardiac hypertrophy, it requires extensive examination by a medical professional. Therefore, VHS using chest radiographic images is mainly used for diagnosing MMVD. However, since the VHS method also requires expert review for determining cardiac hypertrophy, it takes a considerable amount of time and the results may vary depending on the individual performing the measurement.

### SUMMARY

The present invention has been devised to solve the problems of the prior art described above, and one technical problem to be solved is to provide a method and apparatus for generating a contour prediction model trained, based on a training dataset, to output a cardiac contour image and a vertebral body contour image of a specific thoracic vertebral region of a subject animal from a chest radiographic image of the subject animal, and a method and system for determining cardiac hypertrophy in animals using the same.

The technical problems to be achieved by the present invention are not limited to those described above, and other technical problems may be derived from the following description of the present invention.

As a technical solution for solving the above-described technical problem, according to a first aspect of the present invention, a method for creating a cardiac contour prediction model is provided. The method is performed by a processor and includes: generating a training dataset by preprocessing labeled images including cardiac contour images and vertebral body contour images of a specific thoracic vertebral region, which are generated based on chest radiographic images of animals, together with the chest radiographic images; and generating an artificial intelligence model trained to output a cardiac contour image and a vertebral body contour image of a specific thoracic vertebral region of a subject animal from a chest radiographic image of the subject animal, based on the training dataset.

Also, as a technical solution for solving the above-described technical problem, according to a second aspect of the present invention, an apparatus for creating a contour prediction model is provided. The apparatus includes at least one processor, and a memory electrically connected to the processor and storing at least one code executed by the processor. When executed by the processor, the memory causes the processor to: preprocess labeled images including cardiac contour images and vertebral body contour images of a specific thoracic vertebral region, which are generated based on chest radiographic images of animals, together with the chest radiographic images, to generate a training dataset; and generate an artificial intelligence model trained to output a cardiac contour image and a vertebral body contour image of a specific thoracic vertebral region of a subject animal from a chest radiographic image of the subject animal, based on the training dataset.

Also, as a technical solution for solving the above-described technical problem, according to a third aspect of the present invention, a method for determining cardiac hypertrophy in an animal is provided. The method is performed by a processor and includes: acquiring and preprocessing chest radiographic images of animals; extracting a cardiac contour image and a vertebral body contour image of a specific thoracic vertebral region based on the preprocessed chest radiographic image using a cardiac contour prediction model; deriving a cardiac area, a cardiac height, and a vertebral body width of a specific thoracic vertebral region based on the cardiac contour image and the vertebral body contour image of the specific thoracic vertebral region; and determining the presence or absence of cardiac hypertrophy by calculating an estimated cardiac volume based on the cardiac area, the cardiac height, and the vertebral body width of the specific thoracic vertebral region.

Also, as a technical solution for solving the above-described technical problem, according to a fourth aspect of the present invention, a system for determining cardiac hypertrophy in an animal is provided. The system includes at least one processor, and a memory electrically connected to the processor and storing at least one code executed by the processor. When executed by the processor, the memory causes the processor to: acquire and preprocess chest radiographic images of animals; extract a cardiac contour image and a vertebral body contour image of a specific thoracic vertebral region based on the preprocessed chest radiographic image using a contour prediction model; derive a cardiac area, a cardiac height, and a vertebral body width of a specific thoracic vertebral region based on the cardiac contour image and the vertebral body contour image; and determine the presence or absence of cardiac hypertrophy by calculating an estimated cardiac volume based on the cardiac area, the cardiac height, and the vertebral body width of the specific thoracic vertebral region.

According to the technical solutions of the present invention described above, it is possible to easily derive the cardiac contour and the vertebral body contour of a specific thoracic vertebral region of a subject animal from a chest radiographic image of the subject animal using an artificial intelligence model.

In addition, according to the present invention, variables such as a two-dimensional area of the heart, the height of the heart, and the vertebral body width of a specific thoracic vertebral region are derived based on the cardiac contour and the vertebral body contour of the specific thoracic vertebral region of the subject animal, and the presence or absence of cardiac hypertrophy is determined by measuring the cardiac volume using the derived variables. Therefore, cardiac hypertrophy can be determined more quickly and accurately compared to the VHS method, which relies on manual evaluation by a veterinarian.

### BRIEF DESCRIPTION OF THE DRAWINGS

FIG. 1 is a block diagram illustrating a configuration of an apparatus for creating a contour prediction model according to an embodiment of the present invention.

FIG. 2 is a diagram illustrating an example of training an artificial intelligence model by the apparatus for creating a contour prediction model shown in FIG. 1.

FIG. 3 is a diagram illustrating an example of a chest radiographic image and a labeled image before preprocessing.

FIG. 4 is a diagram illustrating an example of a chest radiographic image after preprocessing.

FIG. 5 is an operation flowchart for explaining a method of creating a cardiac contour prediction model according to another embodiment of the present invention.

FIGS. 6 to 9 are flowcharts illustrating detailed steps included in the method of creating a cardiac contour prediction model shown in FIG. 5.

FIG. 10 is a block diagram illustrating a configuration of a system for determining cardiac hypertrophy according to another embodiment of the present invention.

FIG. 11 is a diagram illustrating an example of determining cardiac hypertrophy by the system for determining cardiac hypertrophy shown in FIG. 10.

FIG. 12 is a diagram illustrating an example of calculating a cardiac area, a cardiac height, and a vertebral body width of a specific thoracic vertebral region.

FIG. 13 is an operation flowchart for explaining a method of determining cardiac hypertrophy according to another embodiment of the present invention.

FIGS. 14 and 15 are flowcharts illustrating detailed steps included in the method of determining cardiac hypertrophy shown in FIG. 13.

### DETAILED DESCRIPTION

Hereinafter, the present invention will be described in detail with reference to the accompanying drawings. However, the present invention may be implemented in various different forms and is not limited to the embodiments described herein. In addition, the accompanying drawings are provided to facilitate understanding of the embodiments disclosed in the present specification, and the technical spirit disclosed in the present specification is not limited by the drawings. All terms used herein, including technical and scientific terms, should be interpreted as having meanings that are commonly understood by those skilled in the art to which the present invention pertains. Predefined terms should be additionally interpreted as having meanings consistent with related technical literature and the context of the present disclosure, and unless otherwise defined, should not be interpreted as having overly idealized or restrictive meanings.

In order to clearly illustrate the present invention in the drawings, parts irrelevant to the description have been omitted, and the sizes, shapes, and configurations of components shown in the drawings may be variously modified. Throughout the specification, like or similar reference numerals are used to denote like or similar parts.

In the following description, suffixes such as “module” and “unit” used for components are assigned or used interchangeably merely for ease of description, and do not inherently indicate different meanings or functions. Also, in describing the embodiments disclosed in the present specification, detailed descriptions of well-known related technologies are omitted when it is determined that such details may obscure the gist of the embodiments disclosed herein.

Throughout the present specification, when a part is described as being “connected” (or “coupled”, “contacted”, or “joined”) to another part, this includes not only a direct connection (or coupling, contact, or joining) between the parts, but also an indirect connection (or coupling, contact, or joining) with another element interposed therebetween. In addition, when a part is described as “including” (or “comprising” or “having”) a certain component, unless explicitly stated otherwise, this does not exclude the presence of other components, but rather means that other components may be further included.

Terms indicating ordinal numbers such as “first” and “second” used in the present specification are merely for distinguishing one component from another and do not imply a specific order or relationship between the components. For example, a first component of the present invention may be referred to as a second component, and likewise, the second component may be referred to as the first component. Furthermore, singular expressions used in the present specification are intended to include plural expressions unless clearly stated otherwise.

The communication module described below may include hardware and software necessary for transmitting and receiving signals such as control signals or data signals through wired or wireless connections with other network devices. The memory may store at least one of the information and data input to the communication module, the information and data required to perform functions by the processor, and the data generated according to execution by the processor. The memory should be interpreted to include both non-volatile storage devices that retain stored information even when power is not supplied, and volatile storage devices that require power to retain stored information. In addition to volatile storage devices that require power to retain stored information, the memory may include magnetic storage media or flash storage media, although the scope of the present invention is not limited thereto. The processor may include various types of devices for controlling and processing data. The processor may refer to a data processing device embedded in hardware, having a physically structured circuit for performing functions represented by code or instructions included in a program. In one example, the processor may be implemented as a microprocessor, a central processing unit (CPU), a processor core, a multiprocessor, an application-specific integrated circuit (ASIC), or a field programmable gate array (FPGA), but the scope of the present invention is not limited thereto.

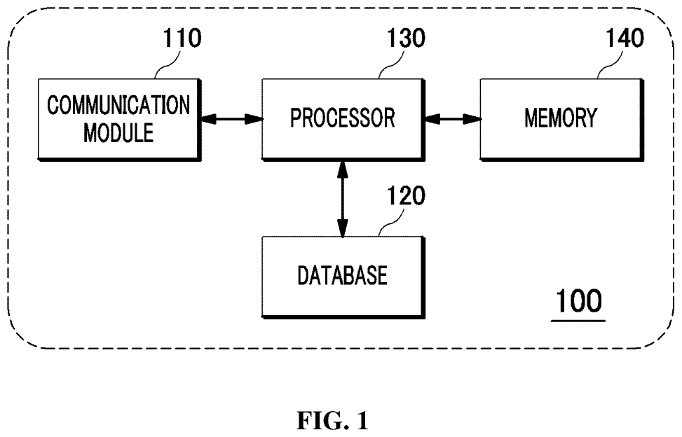

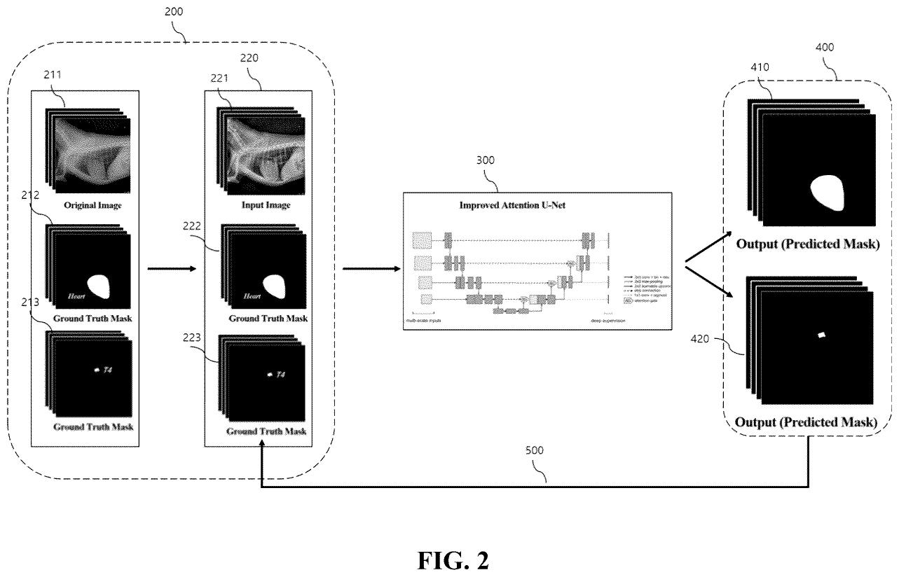

FIG. 1 is a block diagram illustrating a configuration of an apparatus 100 for creating a contour prediction model according to an embodiment of the present invention, and FIG. 2 is a diagram illustrating an example of training an artificial intelligence model by the contour prediction model creation apparatus 100.

Referring to FIGS. 1 and 2 together, the contour prediction model creation apparatus 100 may include at least one processor 130 and a memory 140, and may further include a communication module 110 and a database 120.

The communication module 110 may transmit and receive data required for creating the contour prediction model by performing data transmission and reception with an external device or server.

The database 120 may be a place where data necessary for creating the contour prediction model is stored. The database 120 may be built in a portion of the memory 140 or implemented as separate hardware.

The processor 130 performs operations according to code stored in the memory 140.

The memory 140 is electrically connected to the processor 130 and stores at least one code executed by the processor 130. The memory 140 stores code that causes the processor 130 to perform the following functions and procedures when executed by the processor 130.

The memory 140 stores code that causes a training dataset 220 to be generated by preprocessing labeled images 212 and 213, including a cardiac contour image 212 and a vertebral body contour image 213 of a specific thoracic vertebral region generated based on chest radiographic images 211 of animals, together with the chest radiographic images 211. More specifically, the memory 140 stores code that causes a first chest radiographic image and a first labeled image 222 and 223 to be generated by resizing the chest radiographic image 211 and the labeled images 212 and 213 to a predetermined size. The memory 140 also stores code that causes a second chest radiographic image to be generated by performing histogram equalization on the first chest radiographic image. More specifically, the code that causes the generation of the second chest radiographic image includes code that divides the first chest radiographic image into a plurality of tiles and performs histogram equalization on each of the plurality of tiles. The code that causes the generation of the second chest radiographic image further includes code that, when a specific tile among the histogram-equalized tiles has a number of pixels at a specific pixel value in the histogram falling outside a predetermined range, selects adjacent tiles adjacent to the specific tile, combines the specific tile and the adjacent tiles into a single tile, and performs histogram equalization on the combined single tile. The memory 140 stores code that causes pixel value normalization to be performed on the second chest radiographic image to generate a preprocessed chest radiographic image 221. The animals may be canines or felines. The vertebral body contour image of a specific thoracic vertebral region may be an image of the fourth thoracic vertebra.

The memory 140 stores code that causes a trained artificial intelligence model 300 to be generated based on the training dataset 220, wherein the artificial intelligence model 300 is trained to output a cardiac contour image and a vertebral body contour image of a specific thoracic vertebral region of a subject animal from a chest radiographic image of the subject animal. The artificial intelligence model 300 may be a deep learning model. The artificial intelligence model 300 may be a convolutional neural network including an attention gate and may utilize Attention U-Net.

More specifically, the memory 140 stores code that causes the training dataset 220 to be divided into training data and validation data. The training dataset 220 may be classified into training data, validation data, and test data in a predetermined ratio. The memory 140 stores code that causes a first contour prediction image including a cardiac contour image 410 and a vertebral body contour image 420 of a specific thoracic vertebral region to be derived by applying a transformation function fw including transformation weights w to the chest radiographic images 221 of the training data. The transformation function fw may be a function combining convolution operations, max pooling, deconvolution operations, skip connections, and a sigmoid function, which are operations used in semantic segmentation, considering model complexity.

The memory 140 stores code that causes a first error value to be derived by comparing a first contour prediction image with a labeled image 222, 223 corresponding to the chest radiographic image 221 of the training data. The memory 140 stores code that causes the artificial intelligence model 300 to be trained in a first stage by modifying the transformation weights w so that the first error value is minimized. The modification of the transformation weights w is performed by a backpropagation algorithm.

More specifically, the backpropagation algorithm sets the cost function value (error value) derived by comparing the first contour prediction image obtained by applying a transformation function fw, which includes specific transformation weights w, to the chest radiographic image 221 with the labeled images 222 and 223 corresponding thereto, as the cost function value for the specific transformation weights w. The backpropagation algorithm modifies the transformation weights w by subtracting a value obtained by multiplying the learning rate of the artificial intelligence model and the partial derivative of the cost function with respect to the transformation weights.

If the partial derivative of the cost function is positive, it indicates that the cost function value is increasing with the transformation function fw having the specific transformation weights w. Therefore, the algorithm subtracts a positive value (product of learning rate and partial derivative) from the transformation weights w. If the partial derivative is negative, it indicates that the cost function value is decreasing with the given weights, and thus the algorithm subtracts a negative value (effectively increasing the weights) to continue reducing the cost.

The memory 140 stores the backpropagation algorithm code that modifies the transformation weights w to minimize the first error value. The memory 140 also stores code that causes an optimizer to be used to adjust the transformation weights w according to the learning rate and gradient direction. The optimizer may be an Adam (Adaptive Moment Estimation) optimizer.

The memory 140 stores code that causes the training completion of the artificial intelligence model to be determined based on the validation data using the AI model 300 trained in the first stage. More specifically, the memory 140 stores code that causes a second contour prediction image including a cardiac contour image 410 and a vertebral body contour image 420 of a specific thoracic vertebral region to be derived for the chest radiographic image 221 of the validation data using the AI model 300 trained in the first stage. The memory 140 stores code that causes a second error value to be derived by comparing the second contour prediction image with the labeled images 222 and 223 corresponding to the chest radiographic image 221 of the validation data. The memory 140 also stores code that causes the transformation weights to be modified so that the second error value is minimized.

The memory 140 stores code that causes the training of the artificial intelligence model 300 to be completed when the number of occurrences in which the second error value does not decrease reaches a predetermined number, or when the second error value transitions from a decreasing trend to an increasing trend. More specifically, the memory 140 stores code that causes the training of the artificial intelligence model 300 to be completed when the number of occurrences of the same error value, without further decrease, reaches a predetermined threshold, or when the second error value changes from decreasing to increasing.

Since overfitting may occur in the training process while minimizing the transformation weights w until the cost function is minimized, the memory 140 stores code that causes the training of the artificial intelligence model 300 to be completed when the second error value obtained from the validation data input to the first-stage trained model transitions from a decreasing trend to an increasing trend.

The artificial intelligence model 300 may include a cardiac contour prediction model and a vertebral body contour prediction model. The cardiac contour prediction model refers to an artificial intelligence model trained to extract a cardiac contour image 410 based on the chest radiographic image 221 and the cardiac contour image 222 of the labeled images 222 and 223. The vertebral body contour prediction model refers to an artificial intelligence model trained to extract a vertebral body contour image 420 of a specific thoracic vertebral region based on the chest radiographic image 221 and the vertebral body image 223 of the labeled images 222 and 223.

The labeled images 222 and 223, the cardiac contour image 410, and the vertebral body contour image 420 output by the artificial intelligence model 300 may be binary images composed of pixel values of 0 and 1.

The first error value and the second error value are values calculated through a cost function. The cost function is a function that quantifies the difference between a cardiac contour image 410 and a vertebral body contour image 420 of a specific thoracic vertebral region, which are obtained by applying a transformation function fw including transformation weights w to a preprocessed chest radiographic image 221, and labeled images 222 and 223 that can be regarded as ground truth.

In semantic segmentation of images, a commonly used metric for calculating the similarity between a cardiac contour image 410 and a vertebral body contour image 420 of a specific thoracic vertebral region, which are obtained by applying the transformation function fw to the preprocessed chest radiographic image 221, and the labeled images 222 and 223 regarded as ground truth, is the Dice Score Coefficient (DSC). The Dice Score Coefficient (DSC) may be expressed by the following Equation 1.

$$DSC = \frac{2 \times {{\sum }_{y = 1}}^{h} {{\sum }_{x = 1}}^{w} {I}_{1} ( x , y ) O ( x , y ) + ϵ}{{{\sum }_{y = 1}}^{h} {{\sum }_{x = 1}}^{w} {I}_{1} ( x , y ) + {{\sum }_{y = 1}}^{h} {{\sum }_{x = 1}}^{w} O ( x , y ) + ϵ} \qquad [ Equation 1 ]$$

In Equation 1, I1(x,y) represents the pixel intensity at coordinates (x,y) in the labeled images 222 and 223, and O(x,y) represents the pixel intensity at coordinates (x,y) in the cardiac contour image 410 and vertebral body contour image 420, which are obtained by applying the transformation function fw including the transformation weights w to the preprocessed chest radiographic image 221. The term e is a small value set to prevent the denominator from becoming zero and may be, for example, 0.01 or 0.001 to avoid significantly affecting the calculation.

The Dice Score Coefficient (DSC) has the characteristic of assigning equal importance to false positive and false negative predictions in image segmentation. However, in image segmentation prediction, the importance of false positives and false negatives may differ depending on the characteristics of the image. Therefore, a generalized function of the Dice Score Coefficient (DSC) is required to adjust the relative importance of false positives and false negatives.

The cost function is a function using the Tversky Index (TI) as a generalized form of the Dice Score Coefficient (DSC) to adjust the relative importance of false positives and false negatives. The cost function includes a Tversky Loss (TL) function and a Focal Tversky Loss (FTL) function. The first error value and the second error value are values derived based on at least one of the Focal Tversky Loss (FTL) function and the Tversky Loss (TL) function.

More specifically, in semantic segmentation of images, the artificial intelligence model 300 performs convolution and pooling operations, during which relatively small regions may lose information (features). To compensate for this loss, the artificial intelligence model 300 additionally receives the chest radiographic image (original image), resized to the feature map size of each intermediate layer. As the chest radiographic image is continuously input, the model can prevent the loss of important information in small regions.

The artificial intelligence model 300 calculates the cost function at multiple intermediate layers in a stepwise manner and derives the final error value by summing them. For example, when the multiple intermediate layers consist of four layers, the artificial intelligence model 300 may use the Focal Tversky Loss (FTL) function as the cost function in three layers and the Tversky Loss (TL) function in the final layer. The artificial intelligence model 300 may derive the final cost function value, i.e., the error value, by summing the FTL values obtained from the three layers and the TL value obtained from the final layer.

The Tversky Index (TI) may be expressed by the following Equation 2.

$$TI = \frac{2 \times {{\sum }_{y = 1}}^{h} {{\sum }_{x = 1}}^{w} {I}_{1} ( x , y ) O ( x , y ) + ϵ}{\begin{matrix}{{\sum }_{y = 1}}^{h} {{\sum }_{x = 1}}^{w} {I}_{1} ( x , y ) + α \times {{\sum }_{y = 1}}^{h} {{\sum }_{x = 1}}^{w} {I}_{1} ( x , y ) ( 1 - O ( x , y ) ) + \\ ( 1 - α ) \times {{\sum }_{y = 1}}^{h} {{\sum }_{x = 1}}^{w} ( 1 - {I}_{1} ( x , y ) ) O ( x , y ) + ϵ\end{matrix}} \qquad [ Equation 2 ]$$

In Equation 2, I1(x,y), O(x,y), and ϵ have the same meanings as in Equation 1. The parameter α is a weight assigned to false negatives in image segmentation prediction, and 1-α is a weight assigned to false positives. The value of α is a hyperparameter that is predetermined to determine the relative importance between false positives and false negatives. When α=0.5, the function becomes equivalent to the standard Dice Score Coefficient (DSC). The value of a may be, for example, 0.7 or 0.2.

A larger Tversky Index (TI) value indicates a higher degree of similarity between the cardiac contour image 410 and vertebral body contour image 420 of a specific thoracic vertebral region, which are obtained by applying a transformation function fw including transformation weights w to the preprocessed chest radiographic image 221, and the labeled images 222 and 223 regarded as ground truth. A Tversky Loss (TL) function based on the Tversky Index (TI) may be expressed by the following Equation 3.

$$TL = 1 - TI \qquad [ Equation 3 ]$$

Referring to Equation 3, the value of the Tversky Loss (TL) function may indicate a smaller error as it approaches zero.

The Focal Tversky Loss (FTL) function is a cost function derived from the Tversky Loss (TL) function. The Focal Tversky Loss (FTL) function applies an exponential function to the Tversky Loss (TL) value so that, during the training process of the artificial intelligence model 300, the model focuses more on images with larger Tversky Loss values than on images with smaller Tversky Loss values. The Focal Tversky Loss (FTL) function may be expressed by the following Equation 4.

$$FTL = ( TL ) \hat{} ( 1 / r ) \qquad [ Equation 4 ]$$

In Equation 4, r is a hyperparameter predetermined to determine the degree of amplification of the Tversky Loss (TL) value. The value of r may be, for example, 4/3.

Referring to Equation 4, the Focal Tversky Loss (FTL) function amplifies the Tversky Loss (TL) value by raising it to the power of 1/r, so that the larger the Tversky Loss (TL) value, the more the Focal Tversky Loss (FTL) value is amplified, and the smaller the Tversky Loss (TL) value, the more the FTL value is reduced. By using the FTL value, the artificial intelligence model 300 can focus its training more intensively on images that are more difficult to learn.

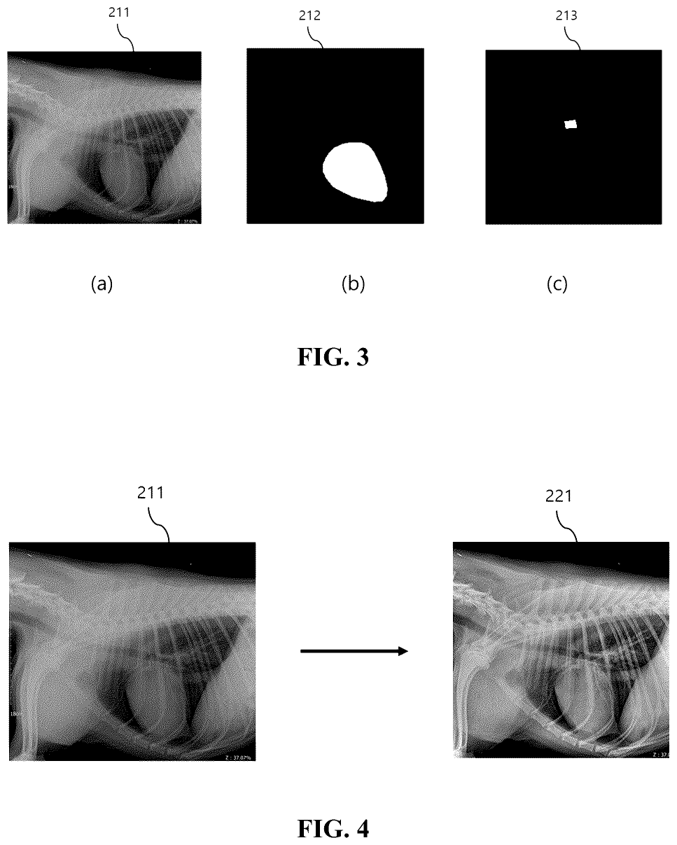

FIG. 3 is a diagram illustrating an example of a training dataset. FIG. 3(a) illustrates a chest radiographic image 211 before preprocessing, and FIGS. 3(b) and 3(c) illustrate labeled images 212 and 213 before preprocessing.

Referring collectively to FIGS. 3(a) through 3(c), the labeled images including a cardiac contour image 212 and a vertebral body contour image 213 of a specific thoracic vertebral region are images generated by a professional such as a veterinarian, who manually marks the cardiac contour and the vertebral body contour of the specific thoracic vertebral region based on the chest radiographic image 211 before preprocessing. The labeled images 212 and 213 may be expressed by the following Equation 5.

$${I}_{1} ( x , y ) ∈ \{ 0 , 1 \} , x ∈ [ 1 , 2 … , w ] , y ∈ [ 1 , 2 … , h ] \qquad [ Equation 5 ]$$

In Equation 5, I1(x,y) represents the pixel intensity at the coordinates (x,y) in the labeled images 212 and 213, w denotes the width of the image, and h denotes the height of the image. Referring to Equation 5, the labeled images 212 and 213 are binary images in which the pixel values, representing pixel intensity, are either 0 or 1. In the cardiac contour image 212 of the labeled image 212 and 213, pixels corresponding to the cardiac contour region have a pixel value of 1, and the background region excluding the cardiac contour region has a pixel value of 0. In the vertebral body contour image 213 of a specific thoracic vertebral region, pixels corresponding to the contour of the vertebral body region have a pixel value of 1, and the background region excluding the vertebral body region has a pixel value of 0.

FIG. 4 is a diagram illustrating an example of a preprocessed chest radiographic image 221.

Referring to FIG. 4, the preprocessed chest radiographic image 221 may be generated by preprocessing the chest radiographic image 211 before preprocessing so that the cardiac and bone contours are enhanced. More specifically, the preprocessed chest radiographic image 221 is generated by resizing the chest radiographic image 211 to a predetermined size, performing histogram equalization and pixel value normalization. The histogram equalization may be performed using a contrast limited adaptive histogram equalization (CLAHE) method. The pixel value normalization is a process of dividing all pixel values of the histogram-equalized chest radiographic image 211 by 255 so that the pixel value range is limited to 0 to 1. The preprocessed chest radiographic image 221 may be expressed by the following Equation 6.

$$0 ≦ {I}_{2} ( x , y ) ≦ 1 , x ∈ [ 1 , 2 … , w ] , y ∈ [ 1 , 2 … , h ] \qquad [ Equation 6 ]$$

In Equation 6, I2(x,y) represents the pixel intensity at the coordinates (x,y) in the preprocessed chest radiographic image 221, and w and h represent the width and height of the chest radiographic image 221, respectively. As shown in Equation 6, the preprocessed chest radiographic image 221 has pixel values in the range of 0 to 1.

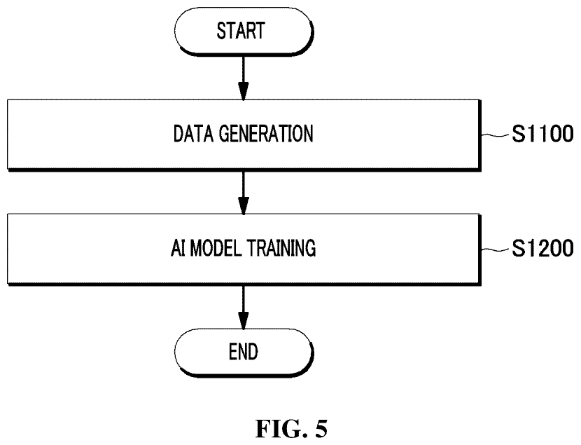

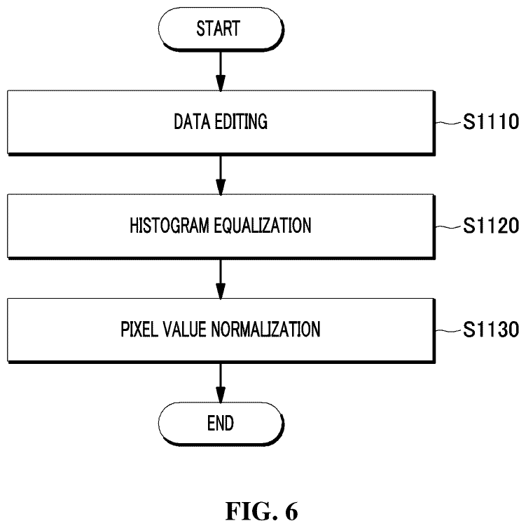

FIG. 5 is an operation flowchart illustrating a method of creating a cardiac contour prediction model according to another embodiment of the present invention, and FIGS. 6 to 9 are flowcharts illustrating detailed steps included in the method of creating the cardiac contour prediction model. Hereinafter, a method of creating a cardiac contour prediction model will be described with reference to FIGS. 6 to 9. Each step of the method of creating a cardiac contour prediction model described below may be performed by the contour prediction model creation apparatus 100 described with reference to FIGS. 1 to 4. Therefore, the contents of the embodiments described with reference to FIGS. 1 to 4 may be equally applied to the following embodiments, and overlapping explanations with the above-described content will be omitted. The steps described below are not necessarily performed in the order listed, and the order of the steps may be configured in various ways, or the steps may be performed substantially simultaneously.

Referring to FIG. 5, the method of creating a cardiac contour prediction model includes a data generation step (S1100) and an artificial intelligence model training step (S1200).

The data generation step (S1100) is a step of generating a training dataset by preprocessing chest radiographic images of animals and labeled images including a cardiac contour image and a vertebral body contour image of a specific thoracic vertebral region generated based on the chest radiographic images. The animals may be canines and felines, and the vertebral body contour image of a specific thoracic vertebral region may be an image of the fourth thoracic vertebra. The artificial intelligence model training step (S1200) is a step of generating an artificial intelligence model trained to output a cardiac contour image and a vertebral body contour image of a specific thoracic vertebral region of a subject animal from a chest radiographic image of the subject animal, based on the training dataset.

Referring to FIG. 6, the data generation step (S1100) includes a data editing step (S1110), a histogram equalization step (S1120), and a pixel value normalization step (S1130).

The data editing step (S1110) is a step of generating a first chest radiographic image and a first labeled image by editing the chest radiographic image and the labeled image to a predetermined size. The histogram equalization step (S1120) is a step of generating a second chest radiographic image by performing histogram equalization on the first chest radiographic image. The pixel value normalization step (S1130) is a step of performing pixel value normalization on the second chest radiographic image.

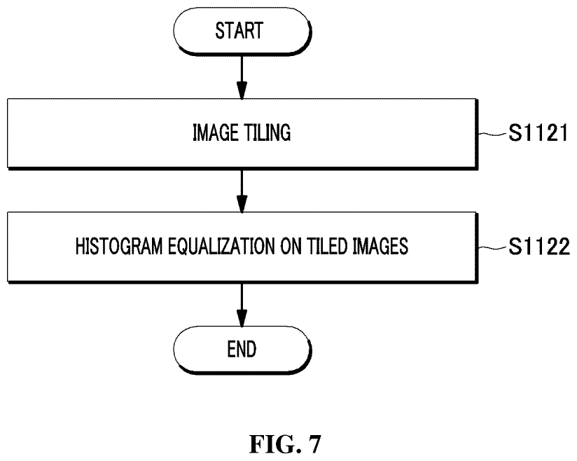

Referring to FIG. 7, the histogram equalization step (S1120) includes an image tiling step (S1121) and a tile-wise histogram equalization step (S1122).

The image tiling step (S1121) is a step of dividing the first chest radiographic image into a plurality of tiles. The tile-wise histogram equalization step (S1122) is a step of performing histogram equalization on each of the plurality of tiles. In the tile-wise histogram equalization step (S1122), if there is a specific tile among the histogram-equalized tiles for which the number of pixels at a specific pixel value in the histogram does not fall within a predetermined range, adjacent tiles neighboring the specific tile are selected, and the specific tile and the adjacent tiles are merged into one tile, and histogram equalization is performed on the merged tile.

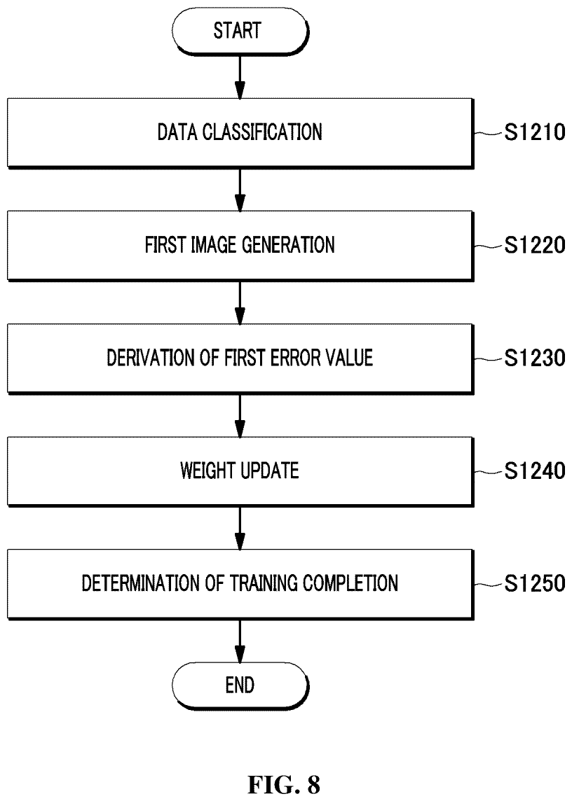

Referring to FIG. 8, the artificial intelligence model training step (S1200) includes a data classification step (S1210), a first image generation step (S1220), a first error value derivation step (S1230), a weight update step (S1240), and a training completion determination step (S1250).

The data classification step (S1210) is a step of classifying the training dataset into training data and validation data. The first image generation step (S1220) is a step of deriving a first contour prediction image including a cardiac contour image and a vertebral body contour image of a specific thoracic vertebral region by applying a transformation function including transformation weights to the chest radiographic images of the training data. The first error value derivation step (S1230) is a step of deriving a first error value by comparing the first contour prediction image with the labeled image corresponding to the chest radiographic image of the training data. The weight update step (S1240) is a step of performing first-stage training of the artificial intelligence model by modifying the transformation weights such that the first error value is minimized. The training completion determination step (S1250) is a step of determining whether the training of the artificial intelligence model is complete, based on the validation data using the first-stage trained artificial intelligence model.

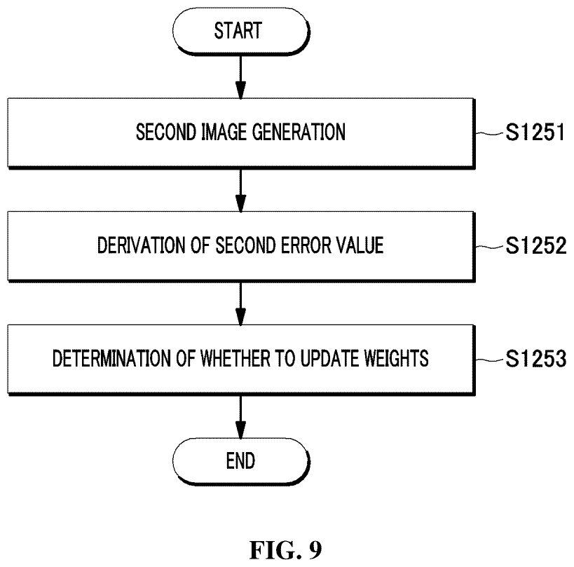

Referring to FIG. 9, the training completion determination step (S1250) includes a second image generation step (S1251), a second error value derivation step (S1252), and a weight update decision step (S1253).

The second image generation step (S1251) is a step of deriving a second contour prediction image including a cardiac contour image and a vertebral body contour image of a specific thoracic vertebral region for the chest radiographic image of the validation data using the first-stage trained artificial intelligence model. The second error value derivation step (S1252) is a step of deriving a second error value by comparing the second contour prediction image with the labeled image corresponding to the chest radiographic image of the validation data. The weight update decision step (S1253) is a step of modifying the transformation weights such that the second error value is minimized. The weight update decision step (S1253) is also a step of completing the training of the artificial intelligence model if the frequency of unchanged second error values meets a predetermined threshold, or if the second error value changes from a decreasing trend to an increasing trend.

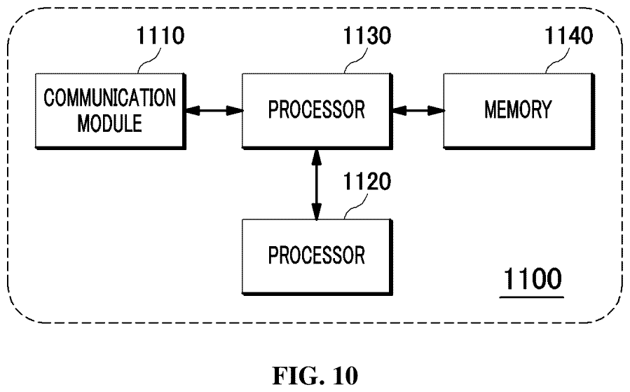

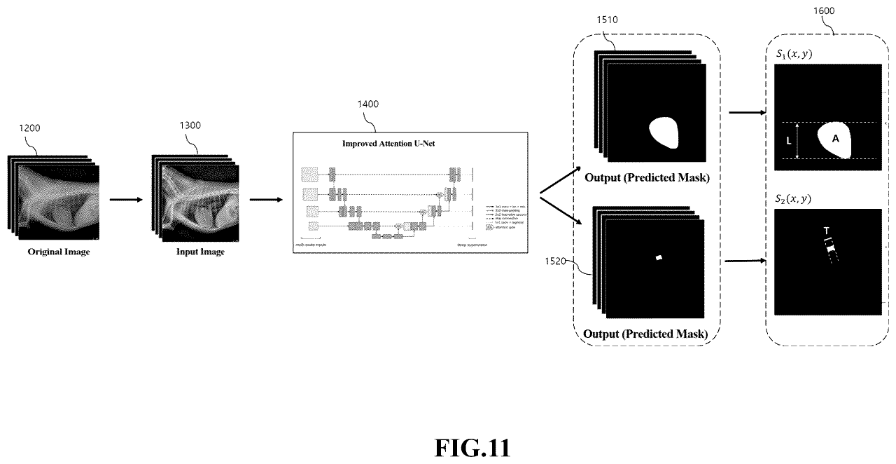

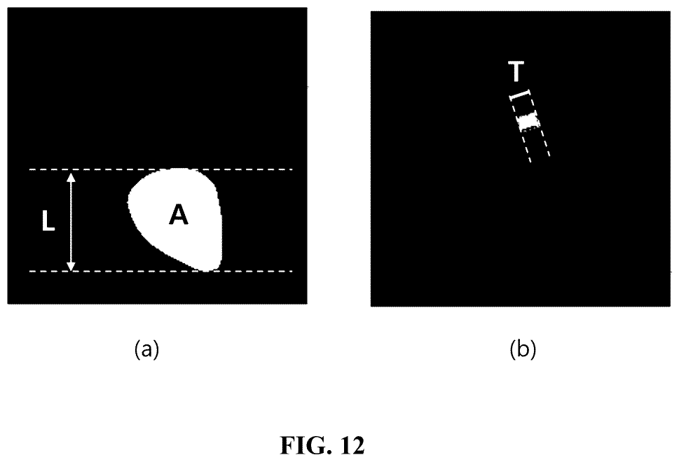

FIG. 10 is a diagram illustrating a cardiac hypertrophy determination system 1100 according to another embodiment of the present invention, and FIG. 11 is a diagram illustrating an example of determining cardiac hypertrophy by the cardiac hypertrophy determination system 1100.

Referring to FIGS. 10 and 11 together, the cardiac hypertrophy determination system 1100 may include at least one processor 1130 and a memory 1140, and may further include a communication module 1110 and a database 1120.

The communication module 1110 may transmit and receive data required for cardiac hypertrophy determination by performing data communication with an external device or server.

The database 1120 may be a place where data required for cardiac hypertrophy determination is stored. The database 1120 may be constructed in a partial area of the memory 1140 or implemented as separate hardware.

The processor 1130 performs operations according to code stored in the memory 1140. The memory 1140 is electrically connected to the processor 1130 and stores at least one code executed by the processor 1140. The memory 1140 stores code that causes the processor 1140 to perform the following functions and procedures when executed.

The memory 1140 stores code that causes chest radiographic images 1200 of animals to be acquired and preprocessed. The animals may be canines or felines. More specifically, the memory 1140 stores code that causes a first chest radiographic image to be generated by resizing the chest radiographic image 1200 to a predetermined size. The memory 1140 also stores code that causes a second chest radiographic image to be generated by performing histogram equalization on the first chest radiographic image. The memory 1140 further stores code that causes pixel value normalization to be performed on the second chest radiographic image.

The memory 1140 stores code that causes a cardiac contour image 1510 and a vertebral body contour image 1520 of a specific thoracic vertebral region to be extracted based on the preprocessed chest radiographic image 1300 using a cardiac contour prediction model 1400. The vertebral body contour image 1520 may be an image of the fourth thoracic vertebra.

The cardiac contour prediction model 1400 includes a first artificial intelligence model and a second artificial intelligence model. The first artificial intelligence model is a model trained, based on a training dataset including training chest radiographic images and corresponding cardiac contour images, to output a cardiac contour image from a given chest radiographic image. The second artificial intelligence model is a model trained, based on a training dataset including training chest radiographic images and corresponding vertebral body contour images of a specific thoracic vertebral region, to output a vertebral body contour image of a specific thoracic vertebral region from a given chest radiographic image.

The weights applied during training of the first artificial intelligence model and the second artificial intelligence model are different. Additionally, the transformation functions and cost functions used in training the first and second artificial intelligence models may also be set differently.

The memory 1140 stores code that causes a cardiac area A, a cardiac height L, and a vertebral body width T of a specific thoracic vertebral region to be derived based on the cardiac contour image 1510 and the vertebral body contour image 1520. The cardiac contour image is a binary image in which pixels corresponding to the cardiac contour region have a value of 1, and background pixels have a value of 0. Similarly, the vertebral body contour image 1520 is a binary image in which pixels corresponding to the vertebral body contour region of a specific thoracic vertebral region have a value of 1, and background pixels have a value of 0.

More specifically, the memory 1140 stores code that causes the cardiac contour region to be identified from the cardiac contour image 1510 and the number of pixels in the region to be used to calculate the cardiac area. The memory 1140 stores code that converts the cardiac contour region into xy coordinates and calculates the cardiac height as the difference between the maximum and minimum Y-axis values.

The memory 1140 stores code that recognizes the region with a pixel value of 1 in the vertebral body contour image 1520 as the vertebral body contour region, and further identifies the minimum-width shape within this region. The memory 1140 stores code that causes the horizontal length of the shape to be calculated as the vertebral body width of the specific thoracic vertebral region.

The memory 1140 stores code that causes an adjusted heart volume index (aHVI) to be calculated based on the cardiac area, cardiac height, and vertebral body width of a specific thoracic vertebral region, and causes the determination of whether cardiac hypertrophy is present based on the calculated aHVI.

The memory 1140 stores code that causes the system to determine whether treatment for cardiac hypertrophy is required when the aHVI falls within a predetermined range. More specifically, the memory 1140 stores code that causes the determination that treatment is required when the aHVI is greater than or equal to 13.5.

The adjusted heart volume index (aHVI) of the present invention is calculated by applying the vertebral body width of a specific thoracic vertebral region to the general cardiac volume estimate, which is calculated by multiplying the cardiac area (A) by the cardiac height (L).

When only the general cardiac volume estimate, calculated by multiplying the cardiac area (A) by the cardiac height (L), is used to determine cardiac hypertrophy, accurate judgment may not be possible because the size of the heart captured in the thoracic radiographic image varies depending on imaging conditions, and the body size of the subject animal also differs from case to case.

Therefore, by applying the vertebral body width (T) of a specific thoracic vertebral region to the general cardiac volume estimate, an adjusted heart volume index (aHVI) can be obtained that allows the determination of cardiac hypertrophy regardless of differences in animal body size or thoracic radiograph imaging conditions.

The adjusted heart volume index (aHVI) may be expressed by the following Equation 7.

$$aHVI = \frac{A \times L}{T \times 10000} \qquad [ Equation 7 ]$$

In Equation 7, A is the cardiac area, L is the cardiac height, and T is the vertebral body width of a specific thoracic vertebral region.

FIG. 12 is a diagram illustrating an example of calculating the cardiac area, cardiac height, and vertebral body width of a specific thoracic vertebral region. FIG. 12(a) illustrates a cardiac contour image, and FIG. 12(b) illustrates a vertebral body image of a specific thoracic vertebral region.

Referring to FIG. 12(a), the cardiac hypertrophy determination system 1100 may recognize a region with a pixel value of 1 in the cardiac contour image as the cardiac contour region and may calculate the cardiac area A based on the number of pixels in the cardiac contour region. The cardiac hypertrophy determination system 1100 may convert the cardiac contour region into XY coordinates and calculate the cardiac height L as the difference between the maximum and minimum Y-coordinate values in the cardiac contour region.

Referring to FIG. 12(b), the cardiac hypertrophy determination system 1100 may recognize the region with a pixel value of 1 in the vertebral body contour image as the vertebral body contour region of a specific thoracic vertebral region and may identify a shape with a minimum area within the vertebral body contour region. The shape with the minimum area may be a rectangle, and more specifically, a rectangular shape.

The cardiac hypertrophy determination system 1100 may set the horizontal length T of the shape as the vertebral body width of the specific thoracic vertebral region. The vertebral body width of the specific thoracic vertebral region may be determined by setting either the longest side or the shortest side of the shape as the horizontal side. The system may then use the length of the selected horizontal side as the vertebral body width T of the specific thoracic vertebral region.

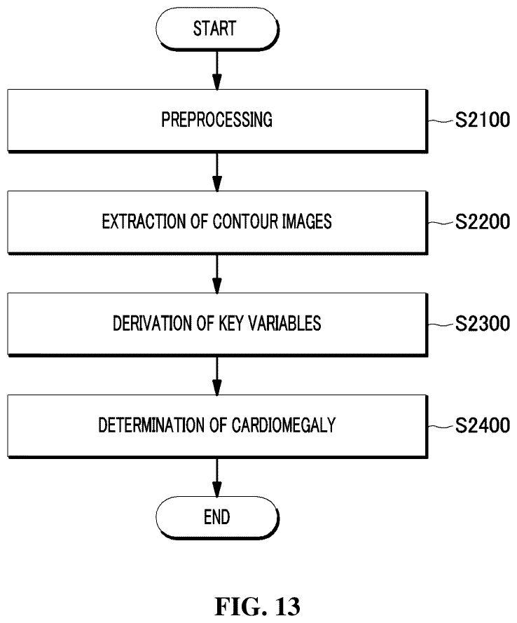

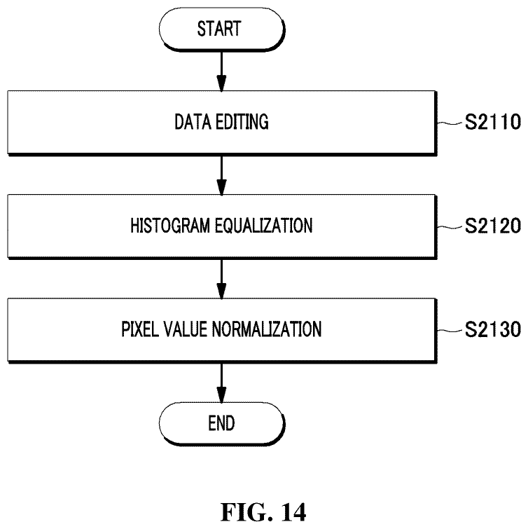

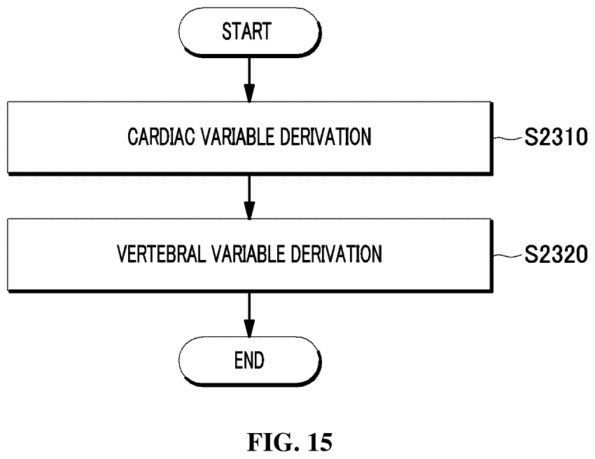

FIG. 13 is an operation flowchart illustrating a method of determining cardiac hypertrophy in an animal according to another embodiment of the present invention, and FIGS. 14 and 15 are flowcharts illustrating detailed steps included in the method of determining cardiac hypertrophy in an animal. Hereinafter, the method of determining cardiac hypertrophy in an animal will be described with reference to FIGS. 13 to 15. Each step of the method of determining cardiac hypertrophy in an animal described below may be performed by the cardiac hypertrophy determination system 1100 described with reference to FIGS. 10 to 12. Therefore, the contents of the embodiments described with reference to FIGS. 10 to 12 may be equally applied to the following embodiments, and overlapping descriptions will be omitted. The steps described below are not necessarily performed in the stated order, and the order of the steps may be configured in various ways, or the steps may be performed substantially simultaneously.

Referring to FIG. 13, the method of determining cardiac hypertrophy in an animal includes a preprocessing step (S2100), a contour image extraction step (S2200), a key variable derivation step (S2300), and a cardiac hypertrophy determination step (S2400).

The preprocessing step (S2100) is a step of acquiring and preprocessing chest radiographic images of animals. The contour image extraction step (S2200) is a step of extracting a cardiac contour image and a vertebral body contour image of a specific thoracic vertebral region from the preprocessed chest radiographic image using a cardiac contour prediction model. The key variable derivation step (S2300) is a step of deriving a cardiac area, a cardiac height, and a vertebral body width of a specific thoracic vertebral region from the cardiac contour image and the vertebral body contour image. The cardiac hypertrophy determination step (S2400) is a step of calculating an adjusted heart volume index (aHVI) based on the cardiac area, the cardiac height, and the vertebral body width, and determining whether cardiac hypertrophy is present. The animals may be canines or felines. The vertebral body contour image of a specific thoracic vertebral region may be an image of the fourth thoracic vertebra.

Referring to FIG. 14, the preprocessing step includes a data editing step (S2110), a histogram equalization step (S2120), and a pixel value normalization step (S2130).

The data editing step (S2110) is a step of generating a first chest radiographic image by resizing the chest radiographic image to a predetermined size. The histogram equalization step (S2120) is a step of generating a second chest radiographic image by performing histogram equalization on the first chest radiographic image. The pixel value normalization step (S2130) is a step of performing pixel value normalization on the second chest radiographic image.

Referring to FIG. 15, the key variable derivation step (S2300) includes a cardiac variable derivation step (S2310) and a vertebral variable derivation step (S2320).

The cardiac variable derivation step (S2310) is a step of identifying the cardiac contour region from the cardiac contour image. The cardiac contour image is a binary image in which the pixels of the cardiac contour region have a value of 1, and the background pixels have a value of 0. The cardiac variable derivation step (S2310) includes a step of calculating the cardiac area based on the number of pixels in the cardiac contour region. It also includes a step of converting the cardiac contour region into XY coordinates and calculating the cardiac height as the difference between the maximum and minimum Y-coordinate values in the region.

The vertebral variable derivation step (S2320) is a step of identifying the vertebral body contour region of a specific thoracic vertebral region. The vertebral body contour image is a binary image in which the vertebral body contour region has a pixel value of 1, and the background has a value of 0. The vertebral variable derivation step (S2320) includes a step of identifying a shape with the minimum area within the vertebral body contour region. It further includes a step of setting the horizontal length of the shape as the vertebral body width of the specific thoracic vertebral region.

What is claimed is:

1. A method of generating a cardiac contour prediction model, wherein each step is performed by a processor, the method comprising:
a) preprocessing chest radiographic images of animals and labeled images including a cardiac contour image and a vertebral body contour image of a specific thoracic vertebral region generated based on the chest radiographic images to generate a training dataset; and
b) generating an artificial intelligence model trained to output a cardiac contour image and a vertebral body contour image of the specific thoracic vertebral region of a subject animal from a chest radiographic image of the subject animal, based on the training dataset.

2. The method of claim 1,
wherein the animals comprise canines and felines, and
wherein the vertebral body contour image of the specific thoracic vertebral region is an image of a fourth thoracic vertebra.

3. The method of claim 1,
wherein the step a) comprises:
a-1) generating a first chest radiographic image and a first labeled image by resizing the chest radiographic image and the labeled image to a predetermined size;
a-2) generating a second chest radiographic image by performing histogram equalization on the first chest radiographic image; and
a-3) performing pixel value normalization on the second chest radiographic image.

4. The method of claim 3,
wherein the step a-2) comprises:
dividing the first chest radiographic image into a plurality of tiles; and
performing histogram equalization on each of the plurality of tiles,
wherein, when a specific tile among the plurality of tiles on which the histogram equalization has been performed includes a number of pixels for a specific pixel value that does not fall within a predetermined range,
adjacent tiles neighboring the specific tile are selected, and the specific tile and the adjacent tiles are merged into a single tile, and the histogram equalization is performed on the merged tile.

5. The method of claim 1,
wherein the step b) comprises:
b-1) classifying the training dataset into training data and validation data;
b-2) deriving a first contour prediction image including a cardiac contour image and a vertebral body contour image of a specific thoracic vertebral region by applying a transformation function including transformation weights to the chest radiographic image of the training data;
b-3) deriving a first error value by comparing the first contour prediction image with the labeled image corresponding to the chest radiographic image of the training data;
b-4) performing first-stage training of the artificial intelligence model by modifying the transformation weights such that the first error value is minimized; and
b-5) determining whether the training of the artificial intelligence model is completed, based on the validation data using the first-stage trained artificial intelligence model.

6. The method of claim 5,
wherein the step b-5) comprises:
deriving a second contour prediction image including a cardiac contour image and a vertebral body contour image of a specific thoracic vertebral region for the chest radiographic image of the validation data using the first-stage trained artificial intelligence model;
deriving a second error value by comparing the second contour prediction image with a labeled image corresponding to the chest radiographic image of the validation data; and
modifying the transformation weights such that the second error value is minimized, and completing the training of the artificial intelligence model when the frequency at which the same second error value is repeatedly derived without decreasing satisfies a predetermined threshold, or when the second error value changes from a decreasing trend to an increasing trend.

7. The method of claim 1,
wherein the artificial intelligence model comprises:
a cardiac contour prediction model trained to extract the cardiac contour image based on the chest radiographic image and the cardiac contour image of the labeled image; and
a vertebral body contour prediction model trained to extract the vertebral body contour image of a specific thoracic vertebral region based on the chest radiographic image and the vertebral body image of the labeled image.

8. The method of claim 1,
wherein the labeled image, the cardiac contour image output by the artificial intelligence model, and the vertebral body contour image of the specific thoracic vertebral region are binary images composed of pixel values of 0 and 1.

9. An apparatus for generating a cardiac contour prediction model, comprising:
at least one processor; and
a memory electrically connected to the processor and storing at least one code executed by the processor,
wherein the memory stores code that, when executed by the processor, causes the processor to:
preprocess chest radiographic images of animals and labeled images including a cardiac contour image and a vertebral body contour image of a specific thoracic vertebral region to generate a training dataset; and
generate an artificial intelligence model trained to output a cardiac contour image and a vertebral body contour image of the specific thoracic vertebral region of a subject animal from a chest radiographic image of the subject animal, based on the training dataset.

10. The apparatus of claim 9,
wherein the memory stores code that causes the processor to:
classify the training dataset into training data and validation data;
derive a first contour prediction image including a cardiac contour image and a vertebral body contour image of a specific thoracic vertebral region by applying a transformation function including transformation weights to the chest radiographic image of the training data;
derive a first error value by comparing the first contour prediction image with a labeled image corresponding to the chest radiographic image of the training data;
perform first-stage training of the artificial intelligence model by modifying the transformation weights such that the first error value is minimized; and
determine whether the training of the artificial intelligence model is completed, based on the validation data using the first-stage trained artificial intelligence model.

11. The apparatus of claim 11,
wherein the memory stores code that causes the processor to:
derive a second contour prediction image including a cardiac contour image and a vertebral body contour image of a specific thoracic vertebral region for the chest radiographic image of the validation data using the first-stage trained artificial intelligence model;
derive a second error value by comparing the second contour prediction image with a labeled image corresponding to the chest radiographic image of the validation data; and
modify the transformation weights such that the second error value is minimized, and complete the training of the artificial intelligence model when the frequency at which the same second error value is repeatedly derived without decreasing satisfies a predetermined threshold.

12. A method of determining cardiac hypertrophy in an animal, wherein each step is performed by a processor, the method comprising:
a) acquiring and preprocessing chest radiographic images of animals;
b) extracting a cardiac contour image and a vertebral body contour image of a specific thoracic vertebral region from the preprocessed chest radiographic image using a cardiac contour prediction model;
c) deriving a cardiac area, a cardiac height, and a vertebral body width of the specific thoracic vertebral region based on the cardiac contour image and the vertebral body contour image; and
d) calculating an adjusted heart volume index based on the cardiac area, the cardiac height, and the vertebral body width of the specific thoracic vertebral region, and determining whether cardiac hypertrophy is present.

13. The method of claim 12,
wherein the animals comprise canines and felines, and
wherein the vertebral body contour image of the specific thoracic vertebral region is an image of a fourth thoracic vertebra.

14. The method of claim 12,
wherein the step a) comprises:
a-1) generating a first chest radiographic image by resizing the chest radiographic image to a predetermined size;
a-2) generating a second chest radiographic image by performing histogram equalization on the first chest radiographic image; and
a-3) performing pixel value normalization on the second chest radiographic image.

15. The method of claim 12,
wherein the cardiac contour image is a binary image in which pixels of a cardiac contour region have a value of 1 and background pixels outside the cardiac contour region have a value of 0, and
wherein the vertebral body contour image of the specific thoracic vertebral region is a binary image in which pixels of the vertebral body contour region have a value of 1 and background pixels outside the vertebral body contour region have a value of 0.

16. The method of claim 15,
wherein the step c) comprises:
recognizing a cardiac contour region from the cardiac contour image;
calculating the cardiac area based on the number of pixels in the cardiac contour region; and
converting the cardiac contour region into XY coordinates and calculating the cardiac height as a difference between the maximum and minimum Y-coordinate values in the region.

17. The method of claim 15,
wherein the step c) comprises:
recognizing a vertebral body contour region of a specific thoracic vertebral region;
identifying a shape with a minimum area within the vertebral body contour region; and
setting a horizontal length of the shape as a vertebral body width of the specific thoracic vertebral region.

18. The method of claim 12,
wherein the cardiac contour prediction model comprises:
a first artificial intelligence model trained to output a cardiac contour image from a specific chest radiographic image, based on a training dataset including training chest radiographic images and cardiac contour images generated based on the training chest radiographic images; and
a second artificial intelligence model trained to output a vertebral body contour image of a specific thoracic vertebral region from a specific chest radiographic image, based on a training dataset including training chest radiographic images and vertebral body contour images of the specific thoracic vertebral region generated based on the training chest radiographic images,
wherein weights applied to training the first artificial intelligence model are different from weights applied to training the second artificial intelligence model.

19. A system for determining cardiac hypertrophy in an animal, comprising:
at least one processor; and
a memory electrically connected to the processor and storing at least one code executed by the processor,
wherein the memory stores code that, when executed by the processor, causes the processor to:
acquire and preprocess chest radiographic images of animals;
extract a cardiac contour image and a vertebral body contour image of a specific thoracic vertebral region from the preprocessed chest radiographic image using a cardiac contour prediction model;
derive a cardiac area, a cardiac height, and a vertebral body width of the specific thoracic vertebral region based on the cardiac contour image and the vertebral body contour image; and
calculate an adjusted heart volume index based on the cardiac area, cardiac height, and vertebral body width of the specific thoracic vertebral region, and determine whether cardiac hypertrophy is present.

20. The system of claim 19,
wherein the memory stores code that causes the processor to:
recognize a region with a pixel value of 1 in the cardiac contour image as a cardiac contour region;
calculate the cardiac area as a number of pixels in the cardiac contour region; and
convert the cardiac contour into XY coordinates and calculate the cardiac height as a difference between the maximum and minimum Y-coordinate values.

21. The system of claim 19,
wherein the memory stores code that causes the processor to:
recognize a region with a pixel value of 1 in the vertebral body contour image as a vertebral body contour region of a specific thoracic vertebral region;
identify a shape with a minimum area within the vertebral body contour region; and
calculate a horizontal length of the shape as a vertebral body width of the specific thoracic vertebral region.
